(19) United States
(12) Patent Application Publication
(10) Pub. No.: US 2006/0257933 A1
(43) Pub. Date: Nov. 16, 2006

(54) Biosensor

(75) Inventors: Taisei Nishimi, Kanagawa (JP); Toshiaki Kubo, Kanagawa (JP); Toshihide Ezoe, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/378,380

(22) Filed: Mar. 20, 2006

(30) Foreign Application Priority Data
Mar. 22, 2005 (JP) 082275/2005

(51) Int. Cl. G01N 33/53; C12M 1/34

(52) U.S. Cl. 435/7.1; 435/287.2

(57) ABSTRACT

It is an object of the present invention to provide a biosensor used to easily produce a hydrogel capable of immobilizing a physically active substance, using safe materials; and a method for producing the same. The present invention provides a biosensor which comprises a substrate having a metal layer on the surface thereof, wherein an acetoacetyl group-containing hydrophilic polymer is bound to said metal layer directly or via an intermediate layer.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

With regard to a detection surface having a functional group capable of immobilizing a physiologically active substance, Japanese Patent No. 2815120 discloses in detail a method for producing a hydrogel, for example. Specifically, a layer of 16-mercaptohexadecanol is allowed to bind to a gold film, so as to form a barrier layer. A hydroxyl group of the barrier layer is treated with epichlorohydrin on the above gold film for epoxy activation. In the next stage, dextran is attached to the barrier layer via an ether bond. Thereafter, bromoacetic acid is allowed to react with the dextran matrix, so as to introduce a carboxymethyl group therein.

As a method for immobilizing a physiologically active substance (for example, a protein or amino acid) having an amino group on the surface of a carboxymethyl-modified dextran that had been produced based on the above method, the following method has been disclosed. Namely, modification can be achieved by treating a portion of the carboxyl group of the carboxymethyl-modified dextran with, for example, an aqueous solution containing N-hydroxylsuccinimide (NHS) and N-(3-dimethylaminopropyl)-N′-ethylcarbodiimide (EDC) hydrochloric acid, such that a reactive ester function occurs. Residual charge, that is, an unreacted carboxyl group, contributes to the concentration of the physiologically active substance onto a detection surface. An aqueous solution containing a physiologically active substance (a protein or amino acid) having an amino group is allowed to come into contact with such a detection surface, so that the physiologically active substance having an amino acid can be bound to a dextran matrix via a covalent bond.

Since a hydrogel produced by the above-mentioned method is able to three-dimensionally immobilize a physiologically active substance having an amino group, it exhibits superior performance as a detection surface of a biosensor. However, a method for producing a hydrogel in accordance with the above-mentioned method is cumbersome and complicated, and it requires a long production time. Also, since it requires the use of a compound such as an epichlorohydrin or a bromoacetic acid, this method has been problematic in terms of safety.

### DISCLOSURE OF INVENTION

It is an object of the present invention to solve the aforementioned problem. That is to say, it is an object of the present invention to provide a biosensor used to easily produce a hydrogel capable of immobilizing a physically active substance, using safe materials; and a method for producing the same.

As a result of intensive studies directed towards achieving the aforementioned object, the present inventors have found that a hydrogel capable of immobilizing a physiologically active substance can be easily produced by using, as a hydrogel used for the detection surface of a biosensor, an acetoacetyl group-containing water-soluble polymer used as a water-soluble adhesive, which acts as a hydrophilic polymer having a reactive functional group that is able to react with an amino group, aldehyde group, or hydrazide group of a physically active substance, thereby completing the present invention.

Thus, the present invention provides a biosensor which comprises a substrate having a metal layer on the surface thereof, wherein a hydrophilic polymer which contains an acetoacetyl group which can be reacted with an amino group, aldehyde group, or hydrazide group of a physically active substance, is bound to said metal layer directly or via an intermediate layer.

Preferably, the acetoacetyl group-containing hydrophilic polymer is acetoacetyl group-containing polyvinyl alcohol.

Preferably, the biosensor according to the present invention is obtained by forming a dense layer on the metal surface using alkanethiol having a reactive group at the terminus thereof, or an oxidant thereof, disulfide, and then allowing the reactive group at the terminus of alkanethiol to react with the acetoacetyl group-containing water-soluble polymer.

Preferably, carboxylic acid is introduced via a reaction of an acetoacetyl group with amino acid.

Preferably, the film thickness of the intermediate layer is between 0.1 and 500 nm.

Preferably, the metal is any of gold, silver, copper, platinum, or aluminum.

Preferably, the biosensor according to the present invention is used in non-electrochemical detection, and more preferably is used in surface plasmon resonance analysis.

Another aspect of the present invention provides a method for producing the biosensor according to the present invention as mentioned above, which comprises a step of allowing an acetoacetyl group-containing water-soluble polymer to chemically bind to the surface of a substrate having a metal layer on the surface thereof directly or via an intermediate layer.

Further another aspect of the present invention provides the biosensor according to the present invention, wherein a physiologically active substance is covalently bound to an acetoacetyl group or to carboxylic acid introduced by allowing an acetoacetyl group to react with amino acid.

Further another aspect of the present invention provides a method for immobilizing a physiologically active substance on a biosensor, which comprises a step of allowing the biosensor according to the present invention to come into contact with a physiologically active substance, thereby binding said physiologically active substance to the surface of said biosensor.

Further another aspect of the present invention provides a method for detecting or measuring a substance interacting with a physiologically active substance, which comprises a step of allowing a test substance to come into contact with the biosensor of the present invention to the surface of which the physiologically active substance binds via a covalent bond.

Preferably, the substance interacting with the physiologically active substance is detected or measured by a non-electrochemical method, and more preferably the substance interacting with the physiologically active substance is detected or measured by surface plasmon resonance analysis.

### BEST MODE FOR CARRYING OUT THE INVENTION

The embodiments of the present invention will be described below.

The biosensor of the present invention is characterized in that it comprises a substrate having a metal layer on the surface thereof, wherein an acetoacetyl group-containing hydrophilic polymer is bound to said metal layer directly or via an intermediate layer.

Hereafter, an acetoacetyl group-containing water-soluble polymer that can be used in the present invention will be explained. An acetoacetyl group-containing water-soluble polymer has the property of reacting with a crosslinking agent having a plurality of aldehyde groups, amino groups, hydrazide groups or the like, at room temperature, so that it becomes hardened. Therefore, it becomes possible to instantly adhere two substrates to each other by applying an acetoacetyl group-containing water-soluble polymer on the surface of one substrate and applying a crosslinking agent on the surface of another substrate, and then by pressing both surfaces. Also, by adding a crosslinking agent to an aqueous solution that contains an acetoacetyl group-containing water-soluble polymer and mixing them, an irrefrangible gel with extremely large water content can be easily obtained at room temperature. JP Patent Publication (Kokai) No. 5-112771 A (1993), JP Patent Publication (Kokai) No. 5-156220 A (1993), JP Patent Publication (Kokai) No. 2002-285117 A, and the like disclose that an acetoacetyl group-containing water-soluble polymer has an excellent property as a water-soluble adhesive.

A method for producing an acetoacetyl group-containing water-soluble polymer will be explained below. Known methods for producing acetoacetylated polyvinyl alcohol, which is a typical compound as an acetoacetyl group-containing water-soluble polymer, include: a method of adding diketene gas to acetic acid in a state where a polyvinyl alcohol resin is dispersed in the acetic acid; a method of adding diketene gas to a solution in which polyvinyl alcohol resin is dissolved in a solvent such as dimethylformamide or dioxane; a method of allowing polyvinyl alcohol powders to directly react with diketene gas; and a method of allowing polyvinyl alcohol to react with an acetoacetic acid ester in a solution for interesterification. The acetoacetylated polyvinyl alcohol used in the present invention can be synthesized by the method described in JP Patent Publication (Kokai) No. 2002-285117 A, for example. Alternatively, commercially available acetoacetylated polyvinyl alcohols such as Gohsefimer Z100, Z200, Z200H, Z210, Z320, or the like, which are made by The Nippon Synthetic Chemical Industry Co., Ltd., can also be used.

Also, an acetoacetyl group-containing water-soluble polymer can be produced via copolymerization of an acetoacetyl group-containing monomer with a water-soluble monomer. Examples of such an acetoacetyl group-containing monomer may include acetoacetoxyethyl acrylate, acetoacetoxyethyl methacrylate, acetoacetoxyethyl crotonate, acetoacetoxypropyl acrylate, acetoacetoxypropyl methacrylate, acetoacetoxypropyl crotonate, 2-cyanoacetoacetoxyethyl methacrylate, N-(2-acetoxyethyl) acrylamide, N-(2-acetoxyaminoethyl) methacrylamide, allyl acetoacetate, and vinyl acetoacetate. These monomers can be produced via a reaction between a functional group-containing ethylene unsaturated monomer and diketene, via a transesterification of the above monomer with an acetoacetoxyalkyl ester, or the like. The acetoacetyl group-containing water-soluble polymer in the invention can be synthesized by the method described in Japanese Patent No. 2777732, for example.

In the present invention, as an acetoacetyl group-containing water-soluble polymer, it is possible to use copolymers with various monomers, other than the aforementioned acetoacetyl group-containing monomer. The monomer unit for such copolymer includes the following monomers:

acrylic acid, methacrylic acid, and their esters: such as acrylic acid, methyl acrylate, butyl acrylate, benzyl acrylate, hydroxyethyl acrylate, CH₂═CHCOO(CH₂CH₂O)ₙR (wherein R is a hydrogen atom and an alkyl group, and n is an integer of 1 or greater), methacrylic acid, methyl methacrylate, ethyl methacrylate, benzylmethacrylate, hydroxyethyl methacrylate, 2-ethylhexyl methacrylate, 2-methoxyethyl methacrylate, N,N-dimethylaminoethyl methacrylate, and 2-sulfoethyl methacrylate;

amides of ethylene unsaturated carboxylic acid: such as acrylamide, methacrylamide, N-acryloyl morpholine, N,N-dimethylacryl amide, and 2-acrylamide-2-methylpropanesulfonic acid (or its salt);

aromatic monomers: such as styrene, vinyl toluene, p-t-butylstyrene, p-vinyl benzoic acid, or vinylnaphthalene; and

other vinyl monomers: such as ethylene, propylene, vinyl chloride, vinylidene chloride, trifluoroethylene, trifluorochloroethylene, vinyl acetate, vinyl propionate, vinyl alcohol, N-vinyl pyrrolidone, N-vinylacetamide, acrylonitrile, or methacrylonitrile.

In the invention, an acetoacetyl group-containing hydrophilic polymer is immobilized on the surface of a biosensor, so as to form a hydrogel thereon. Therefore, it is preferable that such an acetoacetyl group-containing hydrophilic polymer have a hydrophilic group as well as a reactive functional group. Specific examples of such a hydrophilic group may include nonionic groups such as a hydroxyl group or an ethylene glycol group, anionic groups such as a sulfonic group, a carboxylic acid, or a phosphate group, cationic groups such as a quaternary ammonium group or a pyridinium group, and zwitterionic groups such as phosphoryl choline group.

In the invention, monomer units having a hydrophilic group include the following monomers:

monomers having a nonionic group: 2-hydroxyethyl acrylate, 2-hydroxyethyl methacrylate, hydroxypropyl acrylate, hydroxypropyl methacrylate, 2-hydroxy-3-chloropropyl acrylate, β-hydroxyethyl-β′-acryloyloxyethyl phthalate, 1,4-butylene glycol monoacrylate, hydroxystyrene, allyl alcohol, methallyl alcohol, isopropenyl alcohol, 1-butenyl alcohol, or the like;

monomers having an anionic group: vinylsulfonic acid, methallylsulfonic acid, 2-acrylamide-2-methylpropanesulfonic acid, sulfoethyl methacrylate, styrenesulfonic acid, acrylic acid, methacrylic acid, 2-(phosphonoethyloxy)ethyl methacrylate, or the like;

monomers having a cationic group: [2-(acryloyloxy)ethyl]trimethylammonium chloride, [2-(methacryloyloxy)ethyl]trimethylammonium chloride, or the like; and

monomers having a zwitterionic group: [2-(methacryloyloxy)ethyl]dimethyl-(3-sulfopropyl)ammonium hydroxide, 2-[(methacryloyloxy)ethyl]phosphorylcholine, or the like.

By introducing a cationic group or an anionic group into an acetoacetyl group-containing water-soluble polymer, a physiologically active substance having opposite charges can be concentrated onto a detection surface via an electrostatic interaction. In the case of a protein dissolved in a buffer solution having a pH that is higher than an isoelectric point, for example, since such a physiologically active substance is electrostatically concentrated onto a hydrogel surface, to which an acetoacetyl group-containing hydrophilic polymer having a cationic group is bound, it can be effectively bonded to a reactive functional group. On the other hand, in the case of a protein dissolved in a buffer solution having a pH that is lower than an isoelectric point, since such a physiologically active substance is electrostatically concentrated onto a hydrogel surface, to which an acetoacetyl group-containing water-soluble polymer having an anionic group is bound, it becomes possible to allow the physiologically active substance to efficiently react with an acetoacetyl group or a carboxylic acid introduced by allowing such an acetoacetyl group to react with amino acid.

In the present invention, known methods can be applied as a method for binding an acetoacetyl group-containing water-soluble polymer acting as a binding matrix to a metal surface. The following methods can be applied: a method for binding a binding matrix to a metal surface via a hydrophobic polymer explained later in the present specification (see JP Patent Publication (Kokai) No. 2004-271514 A and Japanese Patent Application No. 2004-225130); and a method for binding a binding matrix to a metal surface via a monolayer in which X-R-Y (wherein X binds to metal, and Y binds to the binding matrix) is assembled at high density as disclosed in Japanese Patent No. 2815120. A preferred binding method comprises forming a dense layer on a metal surface using alkanethiol having a reactive group at its terminal (or its oxidant, disulfide), and then allowing a reactive group of the alkanethiol terminal to react with an acetoacetyl group-containing water-soluble polymer.

A physiologically active substance is allowed to come into contact with the surface of the biosensor of the present invention, so that an acetoacetyl group can be covalently bound to the physiologically active substance, thereby enabling immobilization of the physiologically active substance on the biosensor. In addition, an amino acid solution is allowed to come into contact with the surface of the biosensor, so that a reactive functional group of the acetoacetyl group-containing water-soluble polymer can be reacted with an amino group of the amino acid. As a result, the above reactive functional group is converted into carboxylic acid. The biosensor surface having carboxylic acid is activated by a known method, for example, using water-soluble carbodiimide such as 1-(3-dimethylaminopropyl)-3-ethylcarbodiimide (EDC) and N-hydroxysuccinimide (NHS), thereby enabling immobilization of a physiologically active substance having an amino group. Preferred examples of a method for activating carboxylic acid used herein may include: the method described in Japanese Patent Application No. 2004-238396 (namely, a method for forming a carboxylic amide group by activating a carboxyl group that exists on the surface of a substrate using any one of the compounds such as a uronium salt, a phosphonium salt, or triazine derivative, which have a certain structure); and the method described in Japanese Patent Application No. 2004-275012 (namely, a method for forming a carboxylic amide group, which comprises activating a carboxyl group that exists on the surface of a substrate with a carbodiimide derivative or its salt, esterifying it with any one of the compounds such as a nitrogen-containing hetero aromatic compound having a hydroxyl group, a phenol derivative having an electron withdrawing group, or an aromatic compound having a thiol group, and then allowing the resultant to react with amine).

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

In the biosensor of the present invention, a metal surface or metal film can be used as a substrate. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, more preferably between 0.5 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and have excellent workability are preferably used.

In the present invention., an acetoacetyl group-containing hydrophilic polymer is bound to said metal layer directly or via an intermediate layer. As the intermediate layer, a layer composed of a hydrophobic polymer or a self-assembling film, or the like, can be used. The hydrophobic polymer and the self-assembling film are explained below.

A hydrophobic polymer used in the present invention is a polymer having no water-absorbing properties. Its solubility in water (25° C.) is 10% or less, more preferably 1% or less, and most preferably 0.1% or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

A substrate is coated with a hydrophobic polymer according to common methods. Examples of such a coating method may include spin coating, air knife coating, bar coating, blade coating, slide coating, curtain coating, spray method, evaporation method, cast method, and dip method.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24 hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value δ was calculated by the following formula. As the cohesive energy (Ecoh) of each functional group and the mol volume (V), those defined by Fedors were used (R. F. Fedors, Polym. Eng. Sci., 14(2), P147, P472(1974)).
Δ=(ΣEcoh/ΣV)^{1/2}

Examples of the SP values of the hydrophobic polymers and the solvents are shown below;
Solvent: 2-phenoxyethanol:25.3 against polymethylmethacrylate-polystyrene copolymer (1:1):21.0
Solvent: acetonitrile:22.9 against polymethylmethacrylate:20.3
Solvent: toluene:18.7 against polystyrene:21.6

The period for contacting a substrate with a liquid which does not contain the hydrophobic polymer is not particularly limited, and is preferably 1 second to 24hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The coating thickness of a hydrophobic polymer is not particularly limited, but it is preferably between 0.1 nm and 500 nm, and particularly preferably between 1 nm and 300 nm.

Next, the self-assembling film is explained. Sulfur compounds such as thiol and disulfides are spontaneously adsorbed onto a noble metal substrate such as gold, providing a monomolecular-sized ultra thin film. Furthermore, such assembly is referred to as a self-assembling film, because it is shown to have sequences depending on the crystal lattice of a substrate or the molecular structure of admolecules. In the present invention, as a self-assembling compound, compounds such as 7-carboxy-1-heptanethiol, 10-carboxy1-decanethiol, 4,4′-dithiobutyric acid, 11-hydroxy-1-undecanethiol, and 11-amino-1-undecanethiol, can be used.

The biosensor of the present invention preferably has a functional group capable of immobilizing a physiologically active substance on the outermost surface of the substrate. The term “the outermost surface of the substrate” is used to mean “the surface, which is farthest from the substrate,” and more specifically, it means “the surface of a compound applied on a substrate, which is farthest from the substrate.”

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

A biosensor to which a physiologically active substance is immobilized as described above can be used to detect and/or measure a substance which interacts with the physiologically active substance.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example 1

The present example relates to a method for producing a sensor chip, using an acetoacetyl group-containing water-soluble polymer.

(1) Preparation of a Gold Surface Substrate

A film was produced on a glass substrate having a size consisting of 8 mm long, 80 mm wide, and 0.5 mm high, using a parallel plate-type six-inch sputtering apparatus (SH-550, manufactured by Ulvac, Inc.), resulting in a chrome thickness of 1 nm on the substrate and a gold thickness of 50 nm on the chrome. This substrate was treated with Model-208UV-ozone cleaning system (TECHNOVISION INC.) for 30 minutes, so as to produce a gold surface substrate.

(2) Preparation of Sample 1 (Comparative Example)

A hydrogel was produced on the gold surface by applying the method described in Japanese Patent No. 2815120. The gold surface substrate produced in (1) above was set in a Petri dish (inside diameter: 16 cm). Ethanol/water (80/20), in which 5.0 mM 11-hydroxyundecanethiol (manufactured by Dojindo Laboratory Co., Ltd.) had been dissolved, was poured on the surface. The Petri dish was incubated in a shaking incubator at 40° C. for 20 minutes. The surface was washed with 5×50 ml of water, 50 ml of ethanol/water (80/20), and 5×50 ml of water. Furthermore, it was allowed to come into contact with 2.0 ml of an epichlorohydrin solution in 20 ml of 0.4M sodium hydroxide and 20 ml of diethyleneglycol dimethylether. Thereafter, the reaction was carried out in a shaking incubator at 25° C. for 4 hours. The surface was washed with 2×50 ml of ethanol and 5×50 ml of water. A solution prepared by dissolving 13.5 g of dextran (T500, Pharmacia) in 40.5 ml of water and adding 4.5 ml of 1 M sodium hydroxide thereto was poured on the epichlorohydrin-treated surface. Subsequently, after the surface had been incubated in a shaking incubator at 25° C. for 20 hours, it was washed with 15×50 ml of water at 50° C. 3.5 g of bromoacetic acid was dissolved in 27 g of 2 M sodium hydroxide solution, and the obtained solution was poured on the dextran-treated surface. Thereafter, it was incubated in a shaking incubator at 28° C. for 16 hours, and then washed with water. The aforementioned reaction with a bromoacetic acid solution, incubation at 28° C. for 16 hours, and washing with water were repeated once again, so as to obtain sample 1.

(3) Preparation of Sample 2

The gold surface substrate produced in (1) above was set in a Petri dish (inside diameter: 16 cm). Ethanol/water (80/20), in which 4.0 mM 8-hydroxyoctanethiol (manufactured by Dojindo Laboratory Co., Ltd.) and 1.0 mM 11-aminoundecanethiol (manufactured by Dojindo Laboratory Co., Ltd.) had been dissolved, was poured on the surface, and the Petri dish was then incubated in a shaking incubator at 40° C. for 20 minutes. The surface was washed with 5×50 ml of water, 50 ml of ethanol/water (80/20), and 5×50 ml of water. An alkaline solution (pH 10.0, adjusted by addition of NaOH), in which 10% by mass of Gohsefimer Z200H (manufactured by the Nippon Synthetic Chemical Industry Co., Ltd.) that is acetoacetylated polyvinyl alcohol had been dissolved, was poured on the surface. Thereafter, the Petri dish was incubated in a shaking incubator at 60° C. for 16 hours. Thereafter, the surface was washed with 5×50 ml of water, so as to obtain sample 2.

(5) Preparation of Sample 3

1 M glycine aqueous solution (pH 8.5, adjusted by addition of NaOH) was poured on the surface that had been obtained by the same operation as that for sample 2, and the Petri dish was then incubated in a shaking incubator at 60° C. for 16 hours. Thereafter, the surface was washed with 5×50 ml of water, so as to obtain sample 3.

(6) Preparation of Sample 4

1 M 5-aminovaleric acid aqueous solution (pH 8.5, adjusted by addition of NaOH) was poured on the surface that had been obtained by the same operation as that for sample 2, and the Petri dish was then incubated in a shaking incubator at 60° C. for 16 hours. Thereafter, the surface was washed with 5×50 ml of water, so as to obtain sample 4.

### Example 2

The present example relates to immobilization of neutral avidin (manufactured by PIERCE) to the sensor chip obtained in Example 1.

The sensor chips 1, 3, and 4, which had been produced in Example 1, were allowed to come into contact with an aqueous solution containing 0.4 M EDC (1-(3-dimethylaminopropyl)-3-ethylcarbodiimide) and 0.1 M NHS (N-hydroxysuccinimide) for 30 minutes. Thereafter, these chips were washed with an HBS-N buffer (manufactured by Biacore, pH 7.4). The composition of the HBS-N buffer is 0.01 mol/l HEPES (N-2-hydroxyethylpiperazine-N′-2-ethanesulfonic acid) (pH 7.4) and 0.15 mol/l NaCl. Subsequently, the sensor chips 1 to 4 were set to the surface plasmon resonance apparatus of the present invention. Each of the sensor chips was set, such that the center position to which a laser light was irradiated was located in the center in a longitudinal direction, and such that the above position was located at 40 mm from the end in a horizontal direction. By capping the chip with a member made of polypropylene, a cell with a size of 1 mm long (longitudinal direction), 7.5 mm wide (horizontal direction), 1 mm deep, was formed. The inside of the cell was replaced with a neutral avidin solution (100 μg/ml, HBS-N buffer), and it was then left at rest for 30 minutes. Thereafter, it was replaced with an HBS-N buffer. With the foregoing operations, N-avidin was immobilized on the surface of the sensor chip via a covalent bond. With regard to the NHS-esterified sensor chip 1, the amount of change in resonance signal (RU value) between the resonance signal before addition of neutral avidin and the resonance signal 3 minutes after completion of replacement with the HBS-N buffer after addition of the neutral avidin was used as a standard of binding amount. With regard to the sensor chips 2 to 4, the amount of change in resonance signal (RU value) between the resonance signal before addition of neutral avidin and the resonance signal 3 minutes after completion of the replacement with the HBS-N buffer after addition of the neutral avidin (that is, the binding amount of neutral avidin) was evaluated with relative values. The obtained results are shown in Table 1.

### Example 3

The present example relates to the measurement of interaction between the sensor chips 1 to 4 obtained in Example 2, to which neutral avidin has been immobilized, and D-biotin (manufactured by Nacalai tesque).

After completion of the measurement in Example 2, the inside of the cell was replaced with an ethanolamine-HCl solution (1 M, pH 8.5), and the activated COOH group that remained without being reacted with neutral avidin was blocked. Thereafter, the inside of the cell was replaced with D-biotin (1 μg/ml, HBS-N buffer), and it was then left at rest for 10 minutes. Thereafter, it was replaced with an HBS-N buffer.

With regard to the sensor chip 1, the amount of change in resonance signal (RU value) between the resonance signal before addition of D-biotin and the resonance signal 3 minutes after washing was used as a standard of binding amount. With regard to the sensor chips 2 to 4, the amount of change in resonance signal (RU value) between the resonance signal before addition of D-biotin and the resonance signal 3 minutes after washing (that is, the binding amount of D-biotin) was evaluated with relative values. The obtained results are shown in Table 1.

TABLE 1

| | | | | Amount | | |
|---|---|---|---|---|---|---|
| | | Activation | Amount of | of | | |
| Sample | | with | N-avidin | D-biotin | | |
| No. | Sensor surface | EDC/NHS | immobilized | bound | Ratio* | Remarks |
| 1 | Dextran + bromoacetic | Yes | 1.00 | 1.00 | 1.000 | Comparative |
| | acid | | | | | example |
| 2 | Gohsefimer | No | 0.69 | 0.75 | 1.087 | The present |
| | Z200H | | | | | invention |
| 3 | Gohsefimer | Yes | 0.83 | 0.90 | 1.084 | The present |
| | Z200H + Glycine | | | | | invention |
| 4 | Gohsefimer | Yes | 1.12 | 1.21 | 1.080 | The present |
| | Z200H + 5- | | | | | invention |
| | aminovaleric | | | | | |
| | Acid | | | | | |

*Amount of D-biotin bound/amount of N-avidin immobilized

The present example has demonstrated that the sensor chip 2 capable of immobilizing N-avidin can be obtained by an extremely simple method, which comprises forming a dense layer on a metal surface using alkanethiol having an amino group at the terminus thereof and alkanethiol having a hydroxyl group at the terminus thereof, and then allowing the amino group to react with an acetoacetyl group-containing water-soluble polymer, without using unsafe epichlorohydrin or bromoacetic acid. Moreover, it has also been demonstrated that a reactive functional group on the surface of the present sensor chip is allowed to react with amino acid, and it is then activated with EDC/NHS, so that the amount of N-avidin immobilized can be increased (sensor chips 3 and 4). The effect of increasing the amount of N-avidin immobilized obtained in the case of using 5-aminovaleric acid as amino acid (sensor chip 4) was higher than that in the case of using glycine as amino acid (sensor chip 3). Thus, it has been demonstrated that the sensor chip 4 is capable of immobilizing a larger amount of N-avidin than Comparative example (sensor chip 1) is.

The amount of D-biotin bound to these sensor chips, on which N-avidin had been immobilized, is almost proportional to the amount of N-avidin immobilized. Accordingly, it has been demonstrated that a protein immobilized on the sensor chip of the present invention maintains binding ability to a low molecular weight compound.

### EFFECTS OF THE INVENTION

According to the present invention, it became possible to provide: a biosensor used to easily produce a hydrogel capable of immobilizing a physically active substance, using safe materials; and a method for producing the same.

1. A biosensor which comprises a substrate having a metal layer on the surface thereof, wherein an acetoacetyl group-containing hydrophilic polymer is bound to said metal layer directly or via an intermediate layer.

2. The biosensor according to claim 1, wherein the acetoacetyl group-containing hydrophilic polymer is acetoacetyl group-containing polyvinyl alcohol.

3. The biosensor according to claim 1, which is obtained by forming a dense layer on the metal surface using alkanethiol having a reactive group at the terminus thereof, or an oxidant thereof, disulfide, and then allowing the reactive group at the terminus of alkanethiol to react with the acetoacetyl group-containing water-soluble polymer.

4. The biosensor according to claim 1, into which carboxylic acid has been introduced via a reaction of an acetoacetyl group with amino acid.

5. The biosensor according to claim 1, wherein the film thickness of the intermediate layer is between 0.1 and 500 nm.

6. The biosensor according to claim 1, wherein the metal is any of gold, silver, copper, platinum, or aluminum.

7. The biosensor according to claim 1, which is used in non-electrochemical detection.

8. The biosensor according to claim 1, which is used in surface plasmon resonance analysis.

9. A method for producing the biosensor according to claim 1, which comprises a step of allowing an acetoacetyl group-containing water-soluble polymer to chemically bind to the surface of a substrate having a metal layer on the surface thereof directly or via an intermediate layer.

10. The biosensor according to claim 1, wherein a physiologically active substance is covalently bound to an acetoacetyl group or to carboxylic acid introduced by allowing an acetoacetyl group to react with amino acid.

11. A method for immobilizing a physiologically active substance on a biosensor, which comprises a step of allowing the biosensor according to claim 1 to come into contact with a physiologically active substance, thereby binding said physiologically active substance to the surface of said biosensor.

12. A method for detecting or measuring a substance interacting with a physiologically active substance, which comprises a step of allowing a test substance to come into contact with the biosensor of claim 1 to the surface of which the physiologically active substance binds via a covalent bond.

13. The method of claim 12, wherein the substance interacting with the physiologically active substance is detected or measured by a non-electrochemical method.

14. The method of claim 13, wherein the substance interacting with the physiologically active substance is detected or measured by surface plasmon resonance analysis.
